(19) United States
(12) Patent Application Publication
(10) Pub. No.: US 2017/0074849 A1
(43) Pub. Date: Mar. 16, 2017

(54) A TEST STRIP FOR MELAMINE DETECTION

(71) Applicants: THE REGENTS OF THE UNIVERSITY OF CALIFORNIA, Oakland, CA (US); THE UNIVERSITY OF MIAMI, Miami, FL (US)

(72) Inventors: Fang Wei, Los Angeles, CA (US); Na Li, Coral Gables, FL (US)

(21) Appl. No.: 15/123,609

(22) PCT Filed: Mar. 5, 2015
(86) PCT No.: PCT/US2015/018958

§ 371 Date: Sep. 2, 2016

Related U.S. Application Data
(60) Provisional application No. 61/948,938, filed on Mar. 6, 2014.

(51) Int. Cl. G01N 33/04; G01N 21/78

(52) U.S. Cl. CPC G01N 33/04; G01N 21/78; G01N 2021/757

(57) ABSTRACT

Described here is a test strip for detection of melamine, comprising: a support configured for capillary flow of a fluid sample from a first end of the support to a second end of the support that is downstream from the first end; a conjugation pad disposed adjacent to the first end of the support and including nanoparticles configured for suspension in the sample flowing past the conjugation pad, the nanoparticles configured to produce a colorimetric effect when exposed to melamine; and a test portion downstream of the conjugation pad and including a molecular recognition agent immobilized on the support and having an affinity for melamine.

### CROSS-REFERENCE TO RELATED APPLICATION

This application claims the benefit of U.S. Provisional Application No. 61/948,938, filed Mar. 6, 2014, the disclosure of which is incorporated herein by reference in its entirety.

### BACKGROUND

Melamine (1,3,5-triazine-2,4,6-triamine) is an important industrial material that has wide application in plastic engineering. However, because of its high nitrogen content and low cost, it has been illegally added to food products as a fraudulent substitute for protein, thus misleading the standard protein test that is based on the total nitrogen content. High levels of melamine exposure could pose adverse health risks.

Currently, detection of melamine in food involves methods to identify melamine, as well as food sample pretreatment methods that are compatible with detection. Currently prevailing methods include competitive ELISA (Enzyme Linked Immunosorbent Assay) and various chromatography-spectrometry based methods. These methods can be sensitive and reliable. However, they usually involve exhaustive sample pretreatment, expensive equipment, and trained personnel, thus limiting their use as a rapid and low-cost screening tool, especially in remote and underdeveloped areas.

### SUMMARY

Certain embodiments of this disclosure relate to an antibody-free lateral flow test strip, which integrates sample pretreatment and detection, for screening melamine in food or another test sample. Specifically, certain embodiments provide a simple-to-use, low-cost, rapid, sensitive, and specific test strip for detecting melamine in food. This test strip is based on the interaction of melamine with unmodified gold nanoparticles and cyanuric acid on a solid porous substrate (e.g., a membrane). With this test strip, melamine detection can be performed on various food items (e.g., milk, egg, and pet food) without any specialized equipment or complicated data analysis. The detection can be rapid, such as within a few minutes. One example of the application of the test strip is as follows. The test strip is immersed or otherwise contacted with samples to be tested. In some embodiments, samples with melamine contamination will result in two visible lines (test and control lines), while samples without melamine will result in one visible line (control line). In other embodiments, samples with melamine contamination will result in one visible line (test line), while samples without melamine will result in another visible line (control line)

One aspect of some embodiments of this disclosure relates to a test strip for detection of melamine, comprising: a support configured for capillary flow of a fluid sample from a first end of the support to a second end of the support that is downstream from the first end; a conjugation pad disposed adjacent to the first end of the support and comprising nanoparticles configured for suspension in the sample flowing past the conjugation pad, the nanoparticles configured to produce a colorimetric effect when exposed to melamine; and a test portion downstream of the conjugation pad and comprising a molecular recognition agent immobilized on the support and having an affinity for melamine.

In some embodiments, the test strip further comprises a control portion downstream of the test portion and comprising melamine immobilized on the support.

In some embodiments, the nanoparticles comprise at least one metal selected from the group consisting of Au (or gold), Ag (or silver), Fe (or iron), Pt (or platinum), Pd (or palladium), Co (or cobalt), Cu (or copper), Ga (or gallium), Ni (or nickel), Ti (or titanium), W (or tungsten), Rh (or rhodium), and Cr (or chromium). Other transition metals (Groups 3, 4, 5, 6, 7, 8, 9, 10, 11, and 12 transition metals), post-transition metals, and alloys or mixtures of the foregoing metals are contemplated as suitable materials for the nanoparticles. In some embodiments, the nanoparticles comprise gold nanoparticles. In some embodiments, the nanoparticles consist essentially of or consist of gold nanoparticles. In some embodiments, the nanoparticles are substantially or totally free of silver nanoparticles, such as where a percent by weight or number of silver nanoparticles relative to a total weight or number of the nanoparticles is less than or equal to 10%, less than or equal to 5%, less than or equal to 4%, less than or equal to 3%, less than or equal to 2%, less than or equal to 1%, less than or equal to 0.5%, less than or equal to 0.1%, or less than or equal to 0.05%. In some embodiments, the nanoparticles consist essentially of or consist of unmodified metal nanoparticles. In some embodiments, the nanoparticles are substantially or totally free of modified metal nanoparticles, such as where a percent by weight or number of modified metal nanoparticles relative to a total weight or number of the nanoparticles is less than or equal to 10%, less than or equal to 5%, less than or equal to 4%, less than or equal to 3%, less than or equal to 2%, less than or equal to 1%, less than or equal to 0.5%, less than or equal to 0.1%, or less than or equal to 0.05%. In some embodiments, the nanoparticles consist essentially of or consist of unmodified gold nanoparticles. In some embodiments, the nanoparticles are substantially or totally free of modified silver nanoparticles, such as where a percent by weight or number of modified silver nanoparticles relative to a total weight or number of the nanoparticles is less than or equal to 10%, less than or equal to 5%, less than or equal to 4%, less than or equal to 3%, less than or equal to 2%, less than or equal to 1%, less than or equal to 0.5%, less than or equal to 0.1%, or less than or equal to 0.05%.

In some embodiments, the nanoparticles have an average size of about 1 to about 1000 nm, or about 1 nm to about 900 nm, or from about 1 nm to about 800 nm, or from about 1 nm to about 700 nm, or from about 1 nm to about 600 nm, or from about 1 nm to about 500 nm, or from about 1 nm to about 400 nm, or from about 1 nm to about 300 nm, or from about 1 nm to about 200 nm, or from about 1 nm to about 100 nm, or from about 1 nm to about 50 nm, or from about 1 nm to about 25 nm.

In some embodiments, the molecular recognition agent comprises at least one compound selected from the group consisting of cyanuric acid (1,3,5-triazine-2,4,6-triol), acetic acid, oxalic acid, tannin, thymine, uracil, guanine, uric acid, riboflavin, barbituric acid, maleimide, succinimide, diacetamide, glutarimide, and their derivatives. In some embodiments, the molecular recognition agent comprises cyanuric acid. In some embodiments, the molecular recognition agent consists essentially of or consists of cyanuric acid. In some embodiments, the molecular recognition agent is substantially or totally free of p-nitroaniline, such as where a percent by weight or moles of p-nitroaniline relative to a total weight or moles of the molecular recognition agent is less than or equal to 10%, less than or equal to 5%, less than or equal to 4%, less than or equal to 3%, less than or equal to 2%, less than or equal to 1%, less than or equal to 0.5%, less than or equal to 0.1%, or less than or equal to 0.05%.

In some embodiments, the conjugation pad is substantially or totally free of antibodies, such as where a concentration of antibodies in the conjugation pad is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the conjugation pad is substantially or totally free of anti-melamine antibodies, such as where a concentration of anti-melamine antibodies in the conjugation pad is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the conjugation pad is substantially or totally free of silver nanoparticles, such as where a concentration of silver nanoparticles in the conjugation pad is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the conjugation pad is substantially or totally free of modified nanoparticles, such as where a concentration of modified nanoparticles in the conjugation pad is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the conjugation pad is substantially or totally free of modified silver nanoparticles, such as where a concentration of modified silver nanoparticles in the conjugation pad is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm².

In some embodiments, the test portion is substantially free of melamine, such as where a concentration of melamine in the test portion is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the test portion is substantially free of immobilized melamine, such as where a concentration of immobilized melamine in the test portion is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm².

In some embodiments, the test portion is configured such that the presence of melamine in the sample results in the presence of a visual cue (e.g., a visible test line) at the test portion, and the absence of melamine in the sample (or the presence of melamine below a detection threshold) results in the absence of the visual cue at the test portion.

In some embodiments, the test strip is configured to detect 100 parts per million (ppm) or less melamine in the test sample, or 50 ppm or less melamine in the test sample, or 20 ppm or less melamine in the test sample, or 10 ppm or less melamine in the test sample, or 5 ppm or less melamine in the test sample, or 2 ppm or less melamine in the test sample, or 1 ppm or less melamine in the test sample.

Another aspect of some embodiments of this disclosure relates to a test strip for detection of melamine, comprising: a support configured for capillary flow of a fluid sample from a first end of the support to a second end of the support that is downstream from the first end; nanoparticles disposed on the support and configured for suspension in the flowing sample, the nanoparticles configured to bind to melamine when exposed to melamine; and a molecular recognition agent immobilized on the support and configured to bind to melamine to form a first visual cue indicative of the presence of melamine in the sample.

In some embodiments, the test strip further comprises melamine immobilized on the support and configured to bind to the nanoparticles to form a second visual cue indicative of the absence of melamine in the sample.

In some embodiments, the nanoparticles comprise at least one metal selected from the group consisting of Au, Ag, Fe, Pt, Pd, Co, Cu, Ga, Ni, Ti, W, Rh, and Cr. In some embodiments, the nanoparticles comprise gold nanoparticles. In some embodiments, the nanoparticles consist essentially of or consist of gold nanoparticles. In some embodiments, the nanoparticles are substantially or totally free of silver nanoparticles. In some embodiments, the nanoparticles consist essentially of or consist of unmodified metal nanoparticles. In some embodiments, the nanoparticles are substantially or totally free of modified metal nanoparticles. In some embodiments, the nanoparticles consist essentially of or consist of unmodified gold nanoparticles. In some embodiments, the nanoparticles are substantially or totally free of modified silver nanoparticles.

In some embodiments, the nanoparticles have an average size of about 1 to about 1000 nm, or about 1 nm to about 900 nm, or from about 1 nm to about 800 nm, or from about 1 nm to about 700 nm, or from about 1 nm to about 600 nm, or from about 1 nm to about 500 nm, or from about 1 nm to about 400 nm, or from about 1 nm to about 300 nm, or from about 1 nm to about 200 nm, or from about 1 nm to about 100 nm, or from about 1 nm to about 50 nm, or from about 1 nm to about 25 nm.

In some embodiments, the molecular recognition agent comprises at least one compound selected from the group consisting of cyanuric acid, acetic acid, oxalic acid, tannin, thymine, uracil, guanine, uric acid, riboflavin, barbituric acid, maleimide, succinimide, diacetamide, glutarimide, and their derivatives. In some embodiments, the molecular recognition agent comprises cyanuric acid. In some embodiments, the molecular recognition agent consists essentially of or consists of cyanuric acid. In some embodiments, the molecular recognition agent is substantially or totally free of p-nitroaniline.

In some embodiments, the test strip is substantially or totally free of antibodies, such as where a concentration of antibodies in the test strip is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the test strip is substantially or totally free of anti-melamine antibodies, such as where a concentration of anti-melamine antibodies in the test strip is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the test strip is substantially or totally free of silver nanoparticles, such as where a concentration of silver nanoparticles in the test strip is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the test strip is substantially or totally free of modified nanoparticles, such as where a concentration of modified nanoparticles in the test strip is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm². In some embodiments, the test strip is substantially or totally free of modified silver nanoparticles, such as where a concentration of modified silver nanoparticles in the test strip is less than or equal to 10 mg/cm², less than or equal to 1 mg/cm², less than or equal to 100 μg/cm², less than or equal to 10 μg/cm², less than or equal to 1 μg/cm², or less than or equal to 0.1 μg/cm².

In some embodiments, the test strip is configured to detect 100 parts per million (ppm) or less melamine in the test sample, or 50 ppm or less melamine in the test sample, or 20 ppm or less melamine in the test sample, or 10 ppm or less melamine in the test sample, or 5 ppm or less melamine in the test sample, or 2 ppm or less melamine in the test sample, or 1 ppm or less melamine in the test sample.

Another aspect of some embodiments of this disclosure relates to a method for detection of melamine, comprising: providing the test strip described herein; and contacting the test strip with a fluid sample to determine the presence or absence of melamine in the sample.

In some embodiments, the fluid sample is commercially available milk. In some embodiments, the fluid sample is commercially available milk without pretreatment.

In some embodiments, the method further comprises correlating the presence of a visual cue (e.g., a visible test line) at the test portion to the presence of melamine in the sample, or correlating the absence of the visual cue at the test portion to the absence of melamine in the sample. In some embodiments, the method does not involve the use of a spectrometer or turbidimeter.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

FIG. 1 illustrates an example lateral flow chromatography for melamine detection with gold nanoparticles (AuNPs) in a conjugation pad, cyanuric acid (CA) on a test line, and melamine (M) on a control line.

FIG. 2 shows an example melamine test strip based on cyanuric acid and unmodified AuNP. Left: Illustration of the testing procedure. Right: Test results for melamine-negative and melamine-positive samples.

### DETAILED DESCRIPTION

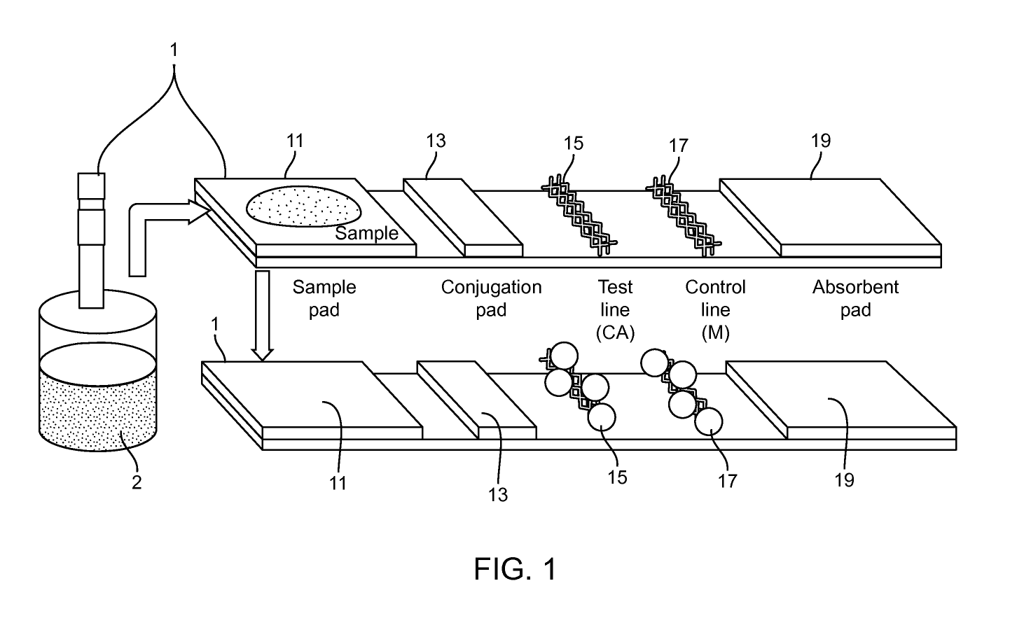

Lateral flow chromatography (LFC) based platform can be used in various analytical and diagnostic applications, including diagnostic kits. To bypass the stability issue of antibodies, a LFC melamine strip test is developed by utilizing the interactions of melamine with non-functionalized or unmodified gold nanoparticles (AuNPs) and cyanuric acid. As shown in FIG. 1, when the strip (1) is immersed in a test sample (2), the fluid will be drawn up via capillary action, sequentially passing through a sample pad (11), a conjugate pad (13) including dispersed AuNPs, a test line (15) or portion including cyanuric acid immobilized or coupled to the strip, and a control line (17) or portion including melamine immobilized or coupled to the strip, and finally reaching an absorbent pad (19). Immobilization of cyanuric acid and melamine to the strip (1) can be through covalent binding, non-covalent binding (e.g., adsorption), or a combination of covalent and non-covalent binding.

When the sample includes melamine, the melamine-AuNP aggregates will be formed in the conjugate pad (13) and then be captured by cyanuric acid on the test line (15), resulting in a colored test line. Other interferents, even those that could cause a color change of AuNP, will not cause a colored test line (or will not cause a noticeable or detectable colored test line); instead, AuNPs will be captured by melamine on the control line (17), thus resulting in a colored control line.

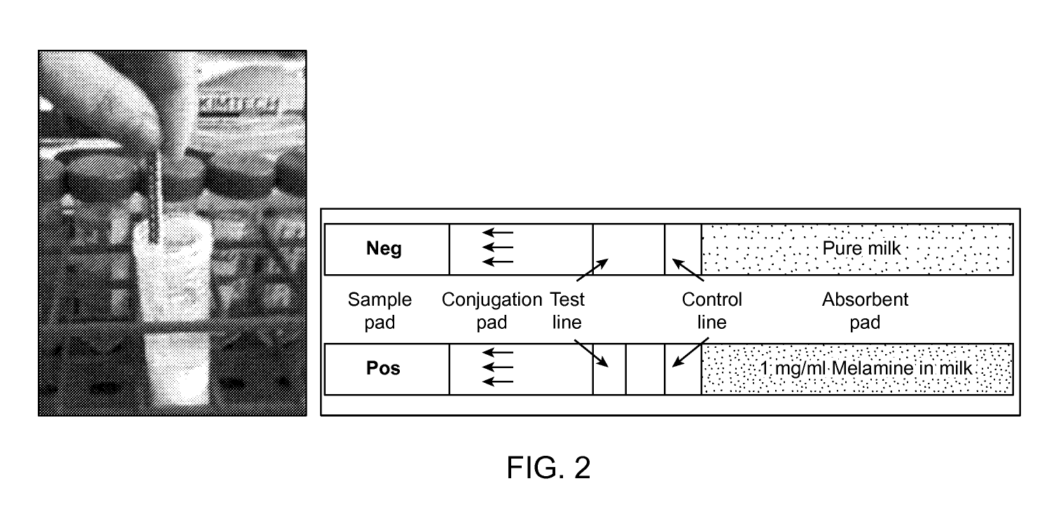

To demonstrate the concept, a prototype of the melamine test strip has been constructed and tested with two milk samples: one spiked with a high concentration of melamine and the other without (pure milk). For each sample, a test strip was dipped in the sample for about 10 seconds (FIG. 2, top) and then allowed to lay substantially horizontally for about 1 minute. As shown in the bottom panel of FIG. 2, on the test strip dipped in the sample including a high concentration of melamine, a purple test line appeared, thus indicating the presence of melamine in the sample. On the other test strip, instead of a purple test line, a purple control line appeared, thus indicating that the test was performed successfully, but no melamine was detected in the sample.

Certain embodiments of a lateral flow test strip for melamine present at least the following advantages: (a) easy-to-use the test strip based platform allows a layperson to perform the test; (b) high sensitivity the AuNP enhanced colorimetric change allows a low level of melamine to be detected; (c) high specificity the highly specific interaction between melamine and cyanuric acid reduces false positives; (d) no requirement for sample pretreatment directly dip the test strip into an untreated, raw sample; (e) low-cost reagents can be mass-produced in a low cost and high stability, thus greatly reducing the costs on manufacturing, packaging, and storage; and/or (f) high robustness and long shelf life antibodies are not required.

Compared to ELISA or chromatography-spectrometry based methods that are often cost- and time-consuming, and involve expensive equipment and trained personnel, the test strip can be readily accessible to a layperson even in resource-limited settings, such as at home and in the field. Compared to other lateral flow based strip tests that rely on surface-immobilized antibodies for molecular recognition and functionalized nanoparticles for visual readout, the test strip utilizes cyanuric acid for molecular recognition and non-functionalized AuNPs for visual readout. Non-functionalized AuNPs and cyanuric acid can be mass-produced with low cost and high stability. This can greatly reduce the costs on manufacturing, packaging, and storage without sacrificing robustness and shelf life. Therefore, the test strip provides a simple-to-use, quick, and economical solution to melamine screening from raw food ingredients and products in low-resource settings.

In some embodiments, a test strip includes nanoparticles (e.g., AuNPs) having an affinity for melamine and that produce a colorimetric effect or signal when contacted or exposed to melamine. Nanoparticles useful in some embodiments can range in average size from about 1 nm to about 1 μm, such as from about 1 nm to about 900 nm, from about 1 nm to about 800 nm, from about 1 nm to about 700 nm, from about 1 nm to about 600 nm, from about 1 nm to about 500 nm, from about 1 nm to about 400 nm, from about 1 nm to about 300 nm, from about 1 nm to about 200 nm, from about 1 nm to about 100 nm, from about 1 nm to about 50 nm, or from about 1 nm to about 25 nm. As used herein, the term “size” refers to a characteristic dimension of an object. Thus, for example, a size of an object that is spherical can refer to a diameter of the object. In the case of an object that is non-spherical, a size of the non-spherical object can refer to a diameter of a corresponding spherical object, where the corresponding spherical object exhibits or has a particular set of derivable or measurable characteristics that are substantially the same as those of the non-spherical object. Thus, for example, a size of a non-spherical object can refer to a diameter of a corresponding spherical object that exhibits optical characteristics that are substantially the same as those of the non-spherical object. Alternatively, or in conjunction, a size of a non-spherical object can refer to an average of various orthogonal dimensions of the object. Thus, for example, a size of an object that is a spheroidal can refer to an average of a major axis and a minor axis of the object. A variety of materials can be used to form nanoparticles, including, for example, metals and metalloids such as Au, Ag, Fe, Pt, Pd, Co, Cu, Ga, Ni, Ti, W, Rh, Cr, and alloys and mixtures thereof. In addition, in certain embodiments, the nanoparticles can be mixed with or formed from other materials, such as polymeric materials and semiconducting materials. The nanoparticles can have a variety of shapes such as spheres, ellipsoids, rods, fibers, discs, tubes, and the like.

As noted above, certain embodiments of a test strip include a molecular recognition agent having an affinity for melamine. As disclosed herein, an example molecular recognition agent is cyanuric acid (1,3,5-triazine-2,4,6-triol). In addition or as an alternative to cyanuric acid, other compounds having a sufficient affinity to bind or capture melamine can be used, such as acetic acid, oxalic acid, tannin, and compounds including certain imide groups such as thymine, uracil, guanine, uric acid, riboflavin, barbituric acid, maleimide, succinimide, diacetamide, glutarimide and their derivatives.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations is not a limitation of the disclosure.

1. A test strip for detection of melamine, comprising:
a support configured for capillary flow of a fluid sample from a first end of the support to a second end of the support that is downstream from the first end;
a conjugation pad disposed adjacent to the first end of the support and comprising nanoparticles configured for suspension in the sample flowing past the conjugation pad, the nanoparticles configured to produce a colorimetric effect when exposed to melamine; and
a test portion downstream of the conjugation pad and comprising a molecular recognition agent immobilized on the support and having an affinity for melamine.

2. The test strip of claim 1, further comprising:
a control portion downstream of the test portion and comprising melamine immobilized on the support.

3. The test strip of claim 1, wherein the nanoparticles comprise at least one metal selected from the group consisting of Au, Ag, Fe, Pt, Pd, Co, Cu, Ga, Ni, Ti, W, Rh, and Cr.

4. The test strip of claim 1, wherein the nanoparticles comprise gold nanoparticles.

5. The test strip of claim 1, wherein the nanoparticles consist essentially of unmodified metal nanoparticles having an average size in a range of 1 nm to 1000 nm.

6. The test strip of claim 1, wherein the molecular recognition agent comprises at least one compound selected from the group consisting of cyanuric acid, acetic acid, oxalic acid, tannin, thymine, uracil, guanine, uric acid, riboflavin, barbituric acid, maleimide, succinimide, diacetamide, glutarimide, and their derivatives.

7. The test strip of claim 1, wherein the molecular recognition agent comprises cyanuric acid.

8. The test strip of claim 1, wherein the conjugation pad is substantially free of anti-melamine antibody.

9. The test strip of claim 1, wherein the test portion is substantially free of melamine.

10. The test strip of claim 1, wherein the presence of melamine in the sample results in the presence of a visual cue at the test portion, and wherein the absence of melamine in the sample results in the absence of a visual cue at the test portion.

11. A test strip for detection of melamine, comprising:
a support configured for capillary flow of a fluid sample from a first end of the support to a second end of the support that is downstream from the first end;
nanoparticles disposed on the support and configured for suspension in the flowing sample, the nanoparticles configured to bind to melamine when exposed to melamine; and
a molecular recognition agent immobilized on the support and configured to bind to melamine to form a first visual cue indicative of the presence of melamine in the sample.

12. The test strip of claim 11, further comprising:
melamine immobilized on the support and configured to bind to the nanoparticles to form a second visual cue indicative of the absence of melamine in the sample.

13. The test strip of claim 11, wherein the nanoparticles comprise at least one metal selected from the group consisting of Au, Ag, Fe, Pt, Pd, Co, Cu, Ga, Ni, Ti, W, Rh, and Cr.

14. The test strip of claim 11, wherein the nanoparticles comprise gold nanoparticles.

15. The test strip of claim 11, wherein the molecular recognition agent comprises at least one compound selected from the group consisting of cyanuric acid, acetic acid, oxalic acid, tannin, thymine, uracil, guanine, uric acid, riboflavin, barbituric acid, maleimide, succinimide, diacetamide, glutarimide, and their derivatives.

16. The test strip of claim 11, wherein the molecular recognition agent comprises cyanuric acid.

17. The test strip of claim 11, wherein the test strip is substantially free of anti-melamine antibody.

18. The test strip of claim 11, wherein the test strip is substantially free of modified nanoparticles.

19. A method for detection of melamine, comprising:
providing the test strip of claim 1; and
contacting the test strip with a fluid sample to determine the presence or absence of melamine in the sample.

20. A method for detection of melamine, comprising:
providing the test strip of claim 1;
contacting the test strip with a fluid sample to determine the presence or absence of melamine in the sample; and
correlating the presence of a visual cue at the test portion to the presence of melamine in the sample, or correlating the absence of a visual cue at the test portion to the absence of melamine in the sample.
